(19) United States
(12) Patent Application Publication
(10) Pub. No.: US 2026/0337138 A1
(43) Pub. Date: Oct. 29, 2026

(54) SEPARATE PARALLEL ZONE HYDROFORMYLATION REACTION

(71) Applicant: JOHNSON MATTHEY DAVY TECHNOLOGIES LIMITED, London (GB)

(72) Inventors: Matthew Thomas Alastair WALTERS, London (GB); Michael Gavin John WILLIAMS, London (GB)

(21) Appl. No.: 19/106,055

(22) PCT Filed: Jul. 21, 2023
(86) PCT No.: PCT/GB2023/051929

§ 371 Date: Feb. 24, 2025

(30) Foreign Application Priority Data
Sep. 26, 2022 (GB) 2213997.6

(51) Int. Cl. C07C 45/50; C07C 29/141; C07C 31/12

(52) U.S. Cl. CPC C07C 45/50; C07C 29/141; C07C 31/12

(57) ABSTRACT

The present invention provides A process for the hydroformylation of olefin to produce normal (N) and iso (I) aldehydes at an N:I ratio R_{A}, the process comprising hydroformylating olefin with hydrogen and carbon monoxide in the presence of ligand-metal catalyst; wherein the hydroformylation is carried out in at least two separate parallel hydroformylation zones, each hydroformylation zone comprising one or more hydroformylation reactors in series; and each separate hydroformylation zone produces N and I aldehydes at a different N:I ratio to the other hydroformylation zone(s); wherein the process comprises the steps of: i) supplying an olefin feed stream into each separate hydroformylation zone; ii) supplying a stream comprising hydrogen and carbon monoxide into each separate hydroformylation zone; iii) recovering an aldehyde product stream from each separate hydroformylation zone; wherein the N:I ratio RA is the total N:I ratio contained in the aldehyde product streams.

### FIELD OF THE INVENTION

The present invention relates to a process for the hydroformylation of olefins to form aldehydes. In particular, the invention relates to controlling the straight chain to branched aldehyde isomer ratio in a hydroformylation process which uses separate hydroformylation zones which produce straight chain and branched aldehydes at different ratios.

### BACKGROUND OF THE INVENTION

The hydroformylation of olefins to produce aldehydes is performed industrially on a large scale. The aldehydes are typically intermediate products in the production of alcohols, acids or esters. A known process for producing such products is the LP Oxo process provided by Dow and Johnson Matthey Davy. In a typical flowsheet, for example as described in U.S. Pat. No. 4,148,830 or U.S. Pat. No. 5,087,763, hydroformylation is performed in the liquid phase using a ligand-rhodium catalyst. A liquid phase reactor effluent is taken from the hydroformylation reactor and fed to a catalyst separation unit where a liquid catalyst solution is separated from the product aldehyde. The liquid catalyst solution is then returned to the reactors. The liquid catalyst solution typically comprises a solvent, rhodium, a ligand, and other components present in the reactor.

Many variations of molecules that can function as a ligand are known. Commercially used ligands are often organomonophosphines, such as triphenylphosphine; organomonophosphites, such as tri methylolpropanephosphite or tris(2,4-di-tert-butylphenyl)phosphite; organopolyphosphites, such as organobisphosphites; or mixtures of any of these. WO2008/115740, WO2011/087690, WO2010/117391 and WO2016/089602 list various ligands. Additionally, organopolyphosphines such as those disclosed in WO2019/231610 may be used. Of these types of ligands, organomonophosphites are believed to be the most active but can have the weakest ligand to rhodium interaction. In the case of, for example, propylene hydroformylation, commercially available organomonophosphites typically produce aldehydes with a relatively low straight chain to branched isomer ratio, for example down to about 0.5 to 1. Commercially available organomonophosphines typically produce aldehydes with a relatively higher straight chain to branched isomer ratio, and somewhat higher still when used in combination with organopolyphosphines. Commercially available organobisphosphites generally have the strongest ligand to rhodium interaction and produce aldehydes with a relatively high straight chain to branched isomer ratio, for example greater than about 20:1.

Often, a high straight chain to branched isomer ratio is desired predominantly because high value downstream products such as plasticizers demand straight chain compounds. This can generally be achieved, for example, using an organobisphosphite ligand during hydroformylation. However, lower ratios of straight to branched chain aldehydes may also be required, for example for use in producing neopentyl glycol. In particular, ratios which lie in between the optimum ratios that can be provided using organomonophosphite or organopolyphosphite alone.

WO2008/115740 discloses a process for controlling the straight chain to branched chain aldehyde ratio using varied ratios of organomonophosphite to organopolyphosphite ligands in the same reaction fluid. However, there can be limitations with such processes particularly if neither ligand-rhodium complex is operating under its optimum performance conditions.

### SUMMARY OF THE INVENTION

The present invention provides a process for the hydroformylation of olefin to produce normal (N) and iso (I) aldehydes at an N:I ratio R_{A}, the process comprising hydroformylating olefin with hydrogen and carbon monoxide in the presence of ligand-metal catalyst;
wherein the hydroformylation is carried out in at least two separate parallel hydroformylation zones, each hydroformylation zone comprising one or more hydroformylation reactors in series; and
each separate hydroformylation zone produces N and I aldehydes at a different N:I ratio to the other hydroformylation zone(s);
wherein the process comprises the steps of:
   i) supplying an olefin feed stream into each separate hydroformylation zone;
   ii) supplying a stream comprising hydrogen and carbon monoxide into each separate hydroformylation zone;
   iii) recovering an aldehyde product stream from each separate hydroformylation zone;
wherein the N:I ratio R_{A} is the total N:I ratio contained in the aldehyde product streams.

Advantageously, loss of metal catalyst during the process can be reduced as compared to processes, such as that disclosed in WO2008/115740, in which the N:I ratio is controlled by using varied ratios of different ligand types in the same reaction fluid. This in turn can have benefits in reduced equipment inventory, capital and running costs. Advantageously, for a particular set of operating conditions and ligand-metal catalyst in each hydroformylation zone, R_{A} can be controlled by the relative flow of the aldehyde product streams from each hydroformylation zone. So, a further advantage of the invention is that the N:I ratio R_{A} can be changed rapidly by changing the flow to each hydroformylation zone unlike in a system such as disclosed in WO2008/115740 in which the N:I ratio is controlled by the ratio of organomonophosphite to organopolyphosphite ligand in the same reaction fluid. It will be understood that substantially the same olefin is being supplied into each separate hydroformylation zone. For example, the olefin feed stream may be divided between the separate hydroformylation zones. Thus, the process may comprise dividing an olefin feed stream into feed streams for each separate hydroformylation zone. The ratio of the flow of the olefin feed stream to each zone may be used as part of the control of the N:I ratio. The flow of the aldehyde product stream from a hydroformylation zone can be controlled by modifying the olefin feed stream, including the relative flow rate of the olefin feed stream to each separate hydroformylation zone, and the stream comprising carbon monoxide and hydrogen. Based on a desired N:I ratio R_{A}, a skilled person can determine, based on the identity of the ligand-metal catalyst used and the operating conditions in each hydroformylation zone, the relative feed flow rates required. In an example, the olefin feed is split and fed to a first hydroformylation zone and a second hydroformylation zone in proportion to the required production capacity of each zone. The stream comprising hydrogen and carbon monoxide is preferably also split into each separate hydroformylation zone in proportion to the required production capacity of each zone.

### BRIEF DESCRIPTION OF THE DRAWINGS

FIG. 1 is a schematic diagram of one aspect of the process of the invention.

FIG. 2 is a schematic diagram of a process of a further aspect of the invention.

FIG. 3 is a chart showing metal loss in a mixed ligand hydroformylation reaction.

FIG. 4 is a chart showing metal loss in a single ligand hydroformylation reaction producing aldehyde with a relatively low N:I ratio.

FIG. 5 is a chart showing metal loss in a single ligand hydroformylation reaction producing aldehyde with a relatively high N:I ratio.

### DETAILED DESCRIPTION OF THE INVENTION

The hydroformylation process, its reagents, conditions, and equipment are known and the hydroformylation step in the present invention can be carried out in accordance with the known techniques employed in conventional hydroformylation processes. The process of the invention uses at least two, preferably two, separate parallel hydroformylation zones. Each hydroformylation zone comprises at least one, typically at least two, typically no more than four, for example two, three or four hydroformylation reactors in series. The hydroformylation zones are parallel in the sense that the reaction fluids in each separate hydroformylation zone do not mix. The reaction fluid comprises a solvent, the ligand-metal catalyst, free ligand, and additional components such as solubilising and stabilising agents. There may be cross-linking of minor streams, for example by passing the vent from a reactor in one hydroformylation zone to a reactor in another hydroformylation zone. A hydroformylation reactor may be a multi-stage reactor such as described in, for example, U.S. Pat. No. 5,763,671 in which there are physical barriers which create one or more theoretical stages per reaction vessel.

The hydroformylation processes in each zone are generally conducted in a continuous manner in which the olefin is hydroformylated with carbon monoxide and hydrogen in a liquid homogenous reaction mixture, i.e. the reaction fluid as described above. Suitable inert solvents include ketones such as acetone, methyl ethyl ketone, methyl isobutyl ketone, acetophenone, and cyclohexanone; aromatics such as benzene, toluene and xylenes; halogenated aromatics including o-dichlorobenzene; ethers such as tetrahydrofuran, dimethoxyethane and dioxane; halogenated paraffins including methylene chloride; paraffinic hydrocarbons such as heptane. The preferred solvent is the aldehyde product and/or the oligomers of the aldehyde product along with the reactive olefin.

The reaction is maintained at temperatures and pressures favourable to the hydroformylation of the olefin and make-up quantities of the olefin, carbon monoxide and hydrogen are supplied to the reaction medium as the reactants are used up. A liquid phase reactor effluent is taken from the hydroformylation reactor(s) in each hydroformylation zone and fed to a catalyst separation and product recovery system in each hydroformylation zone where liquid catalyst solution is separated from product aldehyde. The liquid catalyst solution is then returned to the reactor(s). The liquid catalyst solution typically comprises a solvent, metal, ligand, and other components present in the reaction fluid. Typical flowsheets are described, for example, in U.S. Pat. No. 4,148,830 or U.S. Pat. No. 5,087,763.

Preferably the olefin is a C₃ to C₁₆ olefin, more preferably a C₃ to C₁₂ olefin and most preferably a C₃ olefin. The olefin is preferably a mono-olefin. The olefin is preferably an acyclic olefin, for example a linear olefin or a branched olefin. For example, the olefin may be propylene or normal butene. Preferably, the aldehyde has one more carbon than the olefin. Thus, the aldehyde is preferably a C₄ to C₁₇ aldehyde, more preferably a C₄ to C₁₃ aldehyde and most preferably a C₄ aldehyde. For example, the aldehyde may be butyraldehyde. A skilled person will understand that the aldehyde produced depends on the olefin used.

The stream comprising hydrogen and carbon monoxide can be obtained from any available source and is generally synthesis gas, known as syngas. Typically, the same source stream is used for each hydroformylation zone which is split into two or more individual streams to feed the reactor(s) in each hydroformylation zone. The molar ratio of hydrogen to carbon monoxide can be in the range of and including about 1:10 to about 100:1, generally about 1:10 to about 10:1 or about 2:1 to about 1:2. The feed flow rate will depend on the ligand-metal catalyst, the olefin feed flow rate and other operating conditions. Such flow rates are known and can be readily calculated by a skilled person.

The stream comprising hydrogen and carbon monoxide, as well as the olefin feed, typically each pass through respective purification systems which help protect the hydroformylation catalytic systems from low levels of impurities such as sulphides and chlorides, such systems are known to a skilled person.

The catalyst metal is generally a transition metal, typically selected from rhodium, cobalt, iridium, ruthenium, iron, nickel, palladium, platinum, osmium, chromium, molybdenum and tungsten and mixtures thereof. Preferably, the metal is selected from rhodium, cobalt, iridium and ruthenium, more preferably selected from rhodium, cobalt and ruthenium, most preferably the metal is rhodium.

It will be understood that one hydroformylation zone will be operated under conditions to produce an aldehyde product stream having higher N:I ratio than another hydroformylation zone, meaning the other hydroformylation zone is operated to produce an aldehyde product stream having a lower N:I ratio. Each separate hydroformylation zone typically uses a different ligand-metal catalyst, which generally means that the ligand used in each zone is different. The metal will typically be the same in each zone. Typically, the hydroformylation zone operated under conditions to produce an aldehyde product stream having a lower N:I ratio is operated under conditions to produce an aldehyde product stream having an N:I ratio of about 2:1 or less, typically at least about 0.5:1. Generally, this zone will comprise a ligand-metal catalyst in which the ligand is an organomonophosphite ligand. Suitable ligands for this reaction zone are known and described in, for example, WO2008/115740, WO2011/087690, WO2010/117391 and WO2016/089602. Typically, the hydroformylation zone operated under conditions to produce an aldehyde product stream having a higher N:I ratio is operated under conditions to produce an aldehyde product stream having an N:I ratio of at least about 6:1, typically about 35:1 or less. Generally, this zone will comprise a ligand-metal catalyst in which the ligand is an organomonophosphine ligand or an organopolyphosphite ligand, such as an organobisphosphite, or an organopolyphosphine ligand, e.g. an organotetraphosphine ligand. Suitable organomonophosphine or organopolyphosphite, e.g. organobisphosphite, ligands for this reaction zone are known and are described in WO2008/115740, WO2011/087690, WO2010/117391, WO2016/089602 and WO2019/231610.

Each hydroformylation zone produces N and I aldehydes at a different N:I ratio and the N:I ratio R_{A} is the total N:I ratio contained in the aldehyde product streams. Put another way, the combined N:I ratio in the separate aldehyde product streams recovered from each hydroformylation zone. So, R_{A} represents the weighted average output of each hydroformylation zone. Accordingly, for a particular set of operating conditions and ligand-metal catalyst in each hydroformylation zone, R_{A} can be controlled by the relative flow of the aldehyde product streams from each hydroformylation zone. So, a further advantage of the invention is that the N:I ratio R_{A} can be changed rapidly by changing the flow to each hydroformylation zone unlike in a system such as disclosed in WO2008/115740 in which the N:I ratio is controlled by the ratio of organomonophosphite to organopolyphosphite ligand in the same reaction fluid.

The flow of the aldehyde product stream from a hydroformylation zone can be controlled by modifying the olefin feed stream and the stream comprising carbon monoxide and hydrogen. Based on a desired N:I ratio R_{A}, a skilled person can readily determine, based on the identity of the ligand-metal catalyst used and the operating conditions in each hydroformylation zone, the relative feed flow rates required. The process of the present invention can be used to produce aldehydes at an N:I ratio R_{A} in a range in which a significant but minor fraction of the I aldehyde is required whilst the catalysts are operating within optimum performance conditions. For example, when R_{A} is at least about 0.5:1 and no more than about 10:1, typically at least about 1.5:1 and no more than about 6:1. In this range, the process of the invention can be particularly advantageous because this can result in reactor systems in each separate hydroformylation zone that are of similar scale which is the most economically advantaged situation.

N:I ratios can be determined by a variety of methods. One example is analysis of either vapor or liquid streams coming from the reactors or process streams using techniques such as gas chromatography (GC), infrared (IR) or nuclear infrared (NIR). The product N:I ratio can also be determined by flow measurements from a distillation column used to separate the N and I aldehydes.

Within each hydroformylation zone, the operating conditions that typically differ from the other hydroformylation zone(s) are carbon monoxide partial pressure, temperature, metal concentration in the reaction fluid and ligand concentration in the reaction fluid, which includes free ligand and ligand complexed with the metal to provide the ligand-metal catalyst. Particular ligand-metal catalysts will have particularly favoured ranges for these operating conditions. Advantageously, each hydroformylation zone can be operated under optimum performance conditions for the ligand-metal catalyst used in the zone.

The concentration of the metal-ligand catalyst in the reaction fluid of a hydroformylation zone need only be at the minimum amount necessary to provide a metal concentration for catalysing the desired hydroformylation process. Generally, the metal concentration will be at least about 20 ppmw, typically at least about 30 ppmw. Generally, the metal concentration will be less than or equal to 1000 ppmw, typically less than or equal to 600 ppmw. When an organophosphine ligand is used, in particular an organomonophosphine, the metal concentration may typically be in the range of and including 100 to 1000 ppmw, suitably 200 to 600 ppmw. When an organomonophosphite ligand is used, the metal concentration may typically be in the range of and including 20 to 200 ppmw, suitably 30 to 100 ppmw. When an organopolyphosphite ligand is used, in particular an organobisphosphite, the metal concertation may typically be in the range of and including 20 to 200 ppmw, suitably 30 to 100 ppmw. For avoidance of doubt, ppmw means parts per million by weight of the reaction fluid.

The amount of ligand in the reaction fluid, which includes free and complexed forms, will generally be greater than 1 molar equivalent with respect to the metal and can be included at a concentration up to the solubility limit of the ligand in the reaction fluid. The particular amount will depend on the nature of the ligand. When an organophosphine ligand is used, in particular an organomonophosphine, the amount of ligand may typically be in the range of and including about 30 to about 500 molar equivalents with respect to the metal, suitably about 100 to about 200 molar equivalents with respect to the metal. When an organomonophosphite ligand is used, the amount of ligand may typically be in the range of and including about 4 to about 200 molar equivalents with respect to the metal. When an organopolyphosphite ligand is used, in particular an organobisphosphite, the amount of ligand may typically be greater than about 1 equivalent and at most about 200 molar equivalents, suitably at most about 5 molar equivalents with respect to the metal. The amounts of metal and ligand in the reaction fluid can readily be determined by known analytical methods. For example, the metal can be quantised by inductively coupled plasma (ICP) techniques and the ligands can be quantised by 31P NMR or HPLC on aliquots of the reaction fluid.

The reaction conditions of the hydroformylation process in each reactor can vary widely. Generally, the hydroformylation process can be conducted at a reaction temperature greater than about 25 °C, typically greater than about 50 °C. The hydroformylation process can be conducted at a reaction temperature less than about 200 °C, typically less than about 120 °C. When an organophosphine ligand is used, including organomonophosphine and organopolyphosphine, it can be beneficial to carry out the hydroformylation reaction at a temperature in the range of and including about 60 °C to about 130 °C, suitably about 75 °C to about 120 °C When an organomonophosphite ligand is used, it can be beneficial to carry out the hydroformylation reaction at a temperature in the range of and including about 50 °C to about 110 °C, suitably about 65 °C to about 100 °C. When an organopolyphosphite ligand is used, it can be beneficial to carry out the hydroformylation reaction at a temperature in the range of and including about 50 °C to about 110 °C, suitably about 60 °C to about 100 °C.

Generally, the total gas pressure comprising olefinic reactant, carbon monoxide, hydrogen, and any inert lights in a hydroformylation zone reactor can range from about 1 psia (6.9 kPa) to about 10,000 psia (68.9 MPa). Typically, the process may be operated at a total gas pressure comprising olefinic reactant, carbon monoxide, and hydrogen of less than about 2,000 psia (13,800 kPa), and suitably less than about 500 psia (3450 kPa). Where the total gas pressure differs between hydroformylation zones, the vent from a reactor in the hydroformylation zone operating at a higher carbon monoxide partial pressure may advantageously be passed to a reactor in another hydroformylation zone. This can increase the efficiency in usage of the stream comprising hydrogen and carbon monoxide.

When certain ligands are used, e.g. when an organomonophosphite ligand is used, a relatively high carbon monoxide partial pressure is desired. This can increase the stability of the ligand system. Accordingly, when an organomonophosphite ligand is used the hydroformylation process is typically conducted at a carbon monoxide partial pressure in the range of and including about 1 bar to about 20 bar, typically about 3 bar to about 10 bar. When certain other ligands are used a lower carbon monoxide partial pressure can be employed without a significant impact on the stability of the ligand system. This can raise activity of the ligand without a stability trade off. Accordingly, particularly when an organomonophosphine or organopolyphosphite ligand is used, the hydroformylation process is typically conducted at a carbon monoxide partial pressure in in the range of and including about 0.1 bar to about 5 bar, typically about 0.5 bar to 4 bar. The ability to provide the optimum carbon monoxide partial pressure environment to each hydroformylation zone is an advantage of the present invention. When one hydroformylation zone is operating under a higher carbon monoxide partial pressure than another hydroformylation zone, although not exclusively under those operating conditions, the vent from a reactor in the hydroformylation zone operating under a higher carbon monoxide partial pressure can be fed to a reactor in the hydroformylation zone operating under a lower carbon monoxide partial pressure. Advantageously, this can increase efficiency of use of the feed stream comprising hydrogen and carbon monoxide.

After the hydroformylation reaction, the aldehyde product streams can be combined for further processing together, or they can be further processed individually. Further processing includes catalyst recovery as well as separation of aldehyde from unreacted olefin and other impurities using a process known in the art such as that described in WO2017/158315. It is an advantage of the invention that downstream processes using increased carbon monoxide partial pressure during catalyst separation, or a carbon monoxide strip gas, such as those disclosed in WO2016/089602 and WO2020/240194, are not required to maintain catalyst stability for the entire process stream. Such processes can be implemented, on a reduced scale, in only the hydroformylation zone for which they are required, for example in a zone in which an organomonophosphite ligand is used. After such further processing, N and I isomer separation may then be carried out on either each individual aldehyde product stream recovered from each separate hydroformylation zone or on the combined streams. Accordingly, the process may further comprise a step of feeding each separate aldehyde product stream into a single N and I isomer separation zone, and recovering a high N:I ratio stream comprising aldehyde having an N:I ratio greater than N:I ratio R_{A} and a low N:I ratio stream comprising aldehyde having an N:I ratio less than N:I ratio R_{A}. Alternatively, the process may further comprise a step of feeding each separate aldehyde product stream into a separate N and I isomer separation zone each comprising an isomer column, and recovering from each separate N and I isomer separation zone a high N:I ratio stream comprising aldehyde having an N:I ratio greater than the N:I ratio in the aldehyde product stream fed into the zone, and a low N:I ratio stream comprising aldehyde having an N:I ratio less than the N:I ratio in the stream fed into the zone. A low N:I ratio stream typically has an N:I ratio less than about 1:90, suitably less than about 1:99. A high N:I ratio stream typically has an N:I ratio greater than about 90:1, suitably greater than about 99:1. An N and I isomer separation zone contains one or more separation vessels, generally distillation columns, which are known in the art, e.g. as described in WO2017/182780.

Commercially important downstream products are alcohols prepared by hydrogenation of the aldehydes produced by the present process, for example butanol prepared from a propylene feed via N butyraldehyde. Other commercially important downstream products are alkylalcohols, typically 2-alkylalkanols, prepared by aldol condensation of normal aldehydes prepared by the process of the present invention followed by dehydration and hydrogenation. For example, the production of 2-ethylhexanol from N butyraldehyde (from a propylene feed) and the production of 2-propylheptanol from N valeraldehyde (from a butylene feed). Other alkylalcohols, such as neopentyl glycol, may be prepared using an aldol condensation reaction which involves an additional aldehyde, in the case of neopentyl glycol that aldehyde is formaldehyde, followed by dehydration and hydrogenation. The production of neopentyl glycol uses I butyraldehyde.

Accordingly, the invention also provides a process for preparing an alcohol, the process comprising the steps of producing N and I aldehydes at an N:I ratio R_{A} using the process of the present invention, then hydrogenating at least some of the aldehyde to provide the alcohol. Prior to hydrogenation, N and I aldehyde isomer separation may be carried out as described above such that the high N:I ratio stream or the low N:I ratio stream is hydrogenated. Alternatively, the aldehydes may be hydrogenated without aldehyde isomer separation. The hydrogenation step may be operated under any suitable conditions, for example as described in WO2019/197831.

The invention also provides a process for preparing an alkylalkanol, typically a 2-alkylalkanol such as 2-ethylhexanol or 2-propylheptanol, also more substituted alkylalkanols such as neopentyl glycol. The process comprises the steps of producing high and low N:I ratio streams using the process described above, then performing an aldol condensation reaction on one of the high or low N:I ratio streams, followed by dehydration and hydrogenation steps to provide the alkylalcohol. Typically, for example, when a 2-alkylalcohol such as 2-ethylhexanol or 2-propylheptanol is prepared, the high N:I ratio stream will be used, typically having an N:I ratio of greater than about 99:1. In the case of neopentyl glycol, the low N:I ratio stream will be used, typically having an N:I ratio of less than about 1:99.

Such processing steps are known in the art and may be operated under any suitable conditions, for example aldol condensation and dehydration as described in U.S. Pat. Nos. 5,434,313, 6,340,778 and 9,006,495 and hydrogenation as described in WO2018/069714.

The present invention will now be described, by way of example, with reference to the accompanying figures and to the production of I and N butyraldehyde by hydroformylation of propylene and syngas. It will be understood that it is equally applicable to the production of other aldehydes from a suitably alternative olefin feed. It will also be understood by a skilled person that the drawings are diagrammatic and that further items of equipment such as reflux drums, pumps, vacuum pumps, compressors, gas recycle compressors, temperature sensors, pressure relief valves, control valves, flow controllers, level controllers, and the like may be required in a commercial plant. The provision of such ancillary items of equipment forms no part of the present invention and is in accordance with conventional chemical engineering practice.

A schematic diagram illustrating the overall concept of the process of the present invention is set out in FIG. 1. The process has two parallel hydroformylation zones each with a different ligand-metal catalyst system. The ligand-metal catalyst system in zone one produces a low N:I aldehyde product ratio (typically around 1:1), whilst the ligand-metal catalyst system in zone two produces a high N:I aldehyde product ratio (typically around 30:1). The propylene 1 and syngas 2 feeds each pass through their respective purification systems 3 and 4. Following feedstock purification the feeds are split and fed to zone one and zone two in proportion to the required production capacity of each zone. Propylene is split into streams 5A to zone one and 5B to zone two, syngas is split into streams 6A to zone one and 6B to zone two.

Hydroformylation zone one reactors 7 and 8, in this case two in series, operate at temperature and pressure conditions optimised for the first zone ligand-metal catalyst system. The reaction temperature is typically the same in zone one reactors 7 and 8, with the pressure in reactor 8 slightly lower to allow ready transfer of fluids from reactor 7 to reactor 8. Liquid 7L and vapour 7V streams are typically fed separately from reactor 7 to reactor 8. Hydroformylation zone two reactors 9 and 10 operate in a similar manner, but typically at different temperature and pressure conditions to zone one, which are optimised for the second ligand-metal catalyst system.

The propylene and syngas feeds pass through the reactors in both hydroformylation zones. Some of the syngas feed may by-pass the first reactor in each zone (streams 6C and 6D) and be fed to the second reactor. The vent gases from the second reactor streams 8V and 10V are each cooled in respective vent condenser 11 or 12 to maximise the recovery of butyraldehyde product, the vent gas streams 11V and 12V are respectively purged from the system to limit the build-up of inerts. The crude reactor product from hydroformylation zone one streams 8L and 11L then pass to the zone one product recovery system 13.

Here, butyraldehyde is vapourised and condensed under optimal conditions to separate it from the catalyst and a concentrated catalyst solution stream 15 is recycled back to the zone one reactors 7 and 8. Typically, an increased carbon monoxide partial pressure, or a carbon monoxide strip gas, will be used during catalyst separation in this zone such as disclosed in WO2016/089602 and WO2020/240194.

In a similar manner, crude reactor product streams from zone two 10L and 12L are passed to the zone two product recovery system 14, here butyraldehyde is vapourised and condensed to separate it from the catalyst and a concentrated catalyst solution stream 16 recycled back to the zone 2 reactors. Advantageously, an increased carbon monoxide partial pressure, or a carbon monoxide strip gas, will not be required in this zone thus reducing the overall equipment and associated expenditure required as compared to a process in which aldehydes having a similar N:I ratio are produced in a single reaction zone using multiple ligand-metal catalysts in the same zone.

The crude butyraldehyde products from hydroformylation zone one stream 17 and hydroformylation zone two stream 18 can then be combined and passed to a stabilising column 19, equipped with associated reboiler and condenser. Here, lights such as propylene, propane and any dissolved gases are removed in overhead stream 21 before the crude butyraldehyde stream 20 is passed to the isomer column 22, equipped with associated reboiler and condenser. The isomer column separates the crude butyraldehyde into N butyraldehyde stream 23 and I butyraldehyde stream 24 products.

In FIG. 2 syngas rich vent gas stream 11V is passed from zone one to the zone two reactors via reactor 9. Accordingly, some of the residual hydrogen and carbon monoxide can be used in hydroformylation synthesis in zone two. In this case, zone one needs to operate at a slightly higher pressure than zone two. This use of the vent gas can increase the efficiency in usage of the stream comprising hydrogen and carbon monoxide.

### EXAMPLES

Low and high N:I ratio flowsheets, as well as a mixed ligand comparative example reflective of the process disclosed in WO2008/115740 were independently tested using a continuously operating miniplant with three hydroformylation reactors. Each reactor has independent syngas supplies and vents allowing single or multiple reactor testing to be conducted. The unit is also equipped with product separation and catalyst recycle to undertake long term continuous testing.

The catalyst solution is introduced into the reactor system after dissolving in butyraldehyde or another suitable solvent (e.g. toluene, Texanol). Syngas and propylene are then introduced to initiate continuous operation. Process parameters are modified to optimise temperature, pressure, ligand-metal catalyst concentration and throughput. Various analytical techniques known to a skilled person are used to determine composition of all gas and liquid streams. All analytical and field equipment are validated through regular equipment calibrations or checks. This data is then used to determine selectivity information regarding the process. The examples were undertaken using optimised conditions for that individual catalyst system, as described above.

### Mixed Ligand System Comparative Example

The reactors were charged with rhodium metal and an organomonophosphite ligand (Ligand A) and an organobisphosphite ligand (Ligand B). The system was operated for a total of 140 days at conditions primarily optimised for Ligand B, with a desired N:I ratio of 3. The reactor system was operated with a mixed butyraldehyde production rate of 3.0 gmol/h, with required N:I ratio butyraldehyde obtained in two different segments of operation (0-70 days & 120-130 days). The decrease in rhodium concentration across these segments, both measured via ICP, was 0.151 μg Rh lost/g butyraldehyde produced during these two periods. The change in rhodium concentration over time is shown in FIG. 3.

### Parallel Zone Example

Hydroformylation of propene to butyraldehyde with a desired N:I ratio of 3 was carried out in two separate reaction zones, a low N:I zone and a high N:I zone. The desired N:I ratio in each zone and the capacity required in each zone is shown in Table 1 below.

TABLE 1

| | Operating N:I | Capacity | N bal* | |
|---|---|---|---|---|
| | ratio | (gmol/h) | (gmol/h) | I bal |
| Low N:I ratio zone | 1.4 | 2.84 | 1.65 | 1.18 |
| High N:I ratio zone | 30 | 2.16 | 2.09 | 0.07 |
| Total | | | 3.75 | 1.25 |

*bal = butyraldehyde

### Low N:I Zone

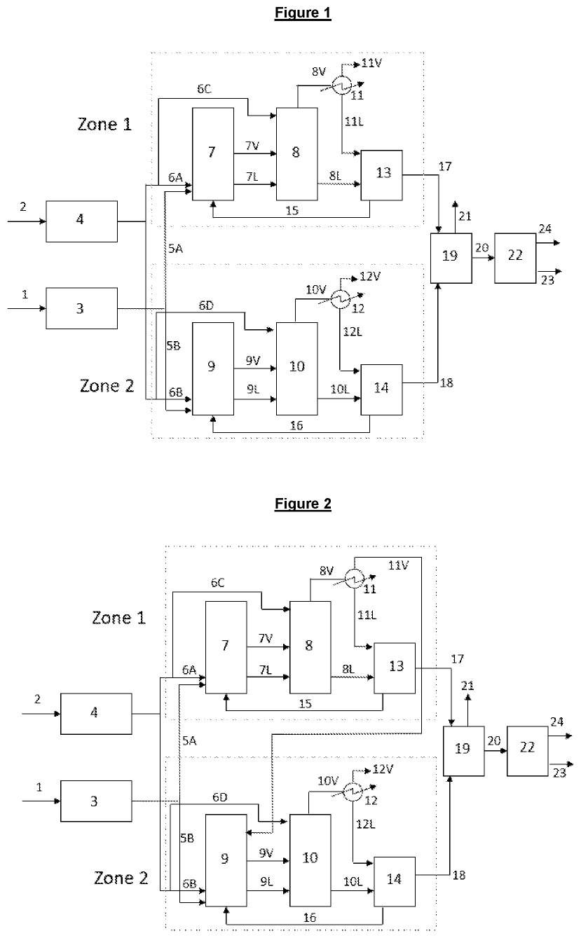

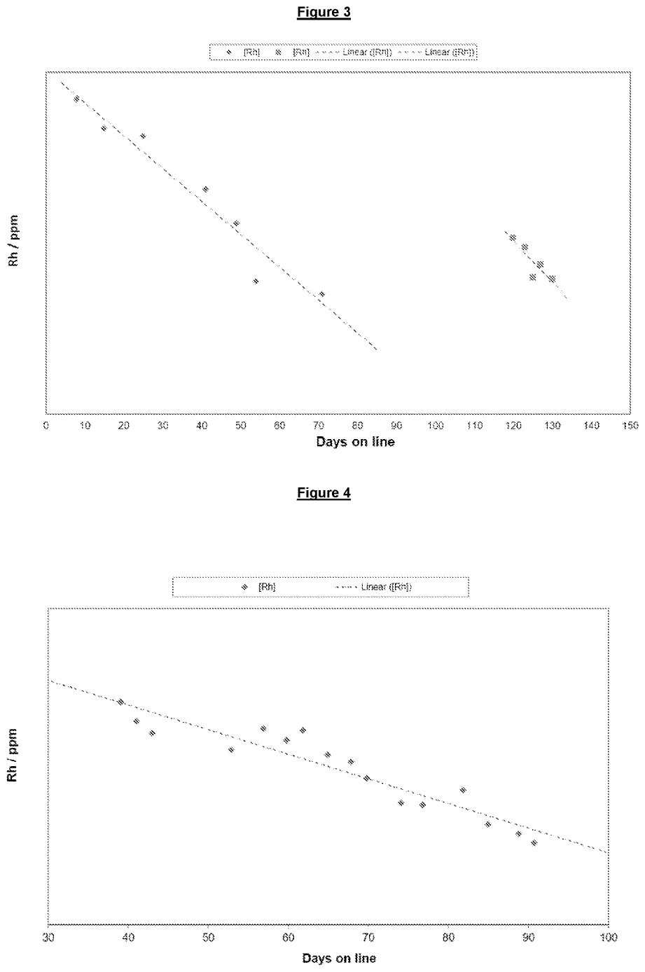

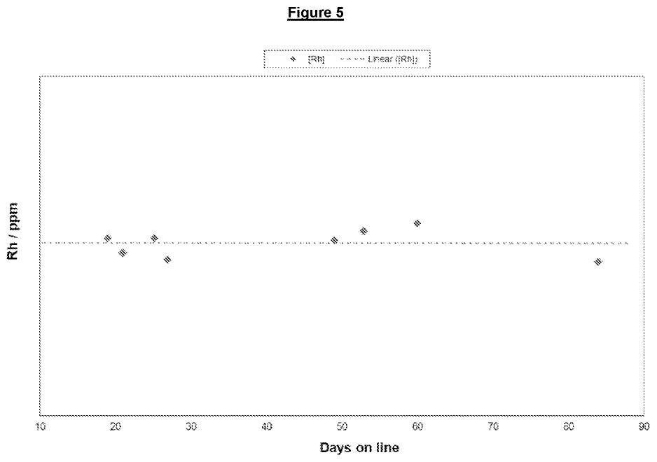

The reactors were charged with rhodium metal and organomonophosphite ligand (Ligand A). The system was operated for a total of 100 days at conditions optimised for Ligand A. The N:I ratio in this example was kept constant around 1.4. The decrease in rhodium concentration, measured via ICP, was 0.073 μg Rh lost/g butyraldehyde produced. The change in rhodium concentration over time is shown in FIG. 4.

High N:I Zone The reactors were charged with rhodium metal and organobisphosphite ligand (Ligand B). The system was operated for 90 days at conditions primarily optimised for Ligand B. The N:I in this example was maintained at around 30:1. The decrease in rhodium concentration, measured via ICP, was 0.002 μg Rh lost/g butyraldehyde produced. The change in rhodium concentration over time is shown in FIG. 5.

### CONCLUSION

Through use of parallel hydroformylation zones for the N:I and relative capacities illustrated in Table 1 above, the mixed ligand system would have an anticipated Rh loss of 0.151 μg Rh lost/g butyraldehyde produced (comparative example), whilst the combined parallel zone system would have an anticipated Rh loss of 0.042 μg Rh lost/g butyraldehyde produced.

1. A process for the hydroformylation of olefin to produce normal (N) and iso (I) aldehydes at an N:I ratio R_{A}, the process comprising hydroformylating olefin with hydrogen and carbon monoxide in the presence of ligand-metal catalyst;
wherein the hydroformylation is carried out in at least two separate parallel hydroformylation zones, each hydroformylation zone comprising one or more hydroformylation reactors in series; and
each separate hydroformylation zone produces N and I aldehydes at a different N:I ratio to the other hydroformylation zone(s);
wherein the process comprises the steps of:
i) supplying an olefin feed stream into each separate hydroformylation zone;
ii) supplying a stream comprising hydrogen and carbon monoxide into each separate hydroformylation zone;
iii) recovering an aldehyde product stream from each separate hydroformylation zone;
wherein the N:I ratio R_{A} is the total N:I ratio contained in the aldehyde product streams.

2. The process of claim 1, wherein the N:I ratio R_{A} is less than about 10:1.

3. The process of claim 1, wherein the N:I ratio R_{A} is at least about 0.5:1.

4. The process of claim 1, wherein the hydroformylation is carried out in two separate parallel hydroformylation zones.

5. The process of claim 1, wherein one hydroformylation zone is operated under conditions to produce an aldehyde product stream having an N:I ratio of about 2:1 or less.

6. The process of claim 5, wherein the hydroformylation zone operated under conditions to produce an aldehyde product stream having an N:I ratio of about 2:1 or less comprises a ligand-metal catalyst in which the ligand is an organomonophosphite ligand.

7. The process of claim 1, wherein one hydroformylation zone is operated under conditions to produce an aldehyde product stream having an N:I ratio of at least about 6:1.

8. The process of claim 7, wherein the hydroformylation zone operated under conditions to produce an aldehyde product stream having an N:I ratio of at least about 6:1 comprises a ligand-metal catalyst in which the ligand is an organomonophosphine ligand.

9. The process of claim 7, wherein the hydroformylation zone operated under conditions to produce an aldehyde product stream having an N:I ratio of at least about 6:1 comprises a ligand-metal catalyst in which the ligand is an organopolyphosphite ligand.

10. A process according to claim 1, wherein the vent from a reactor in a hydroformylation zone operating under a higher carbon monoxide partial pressure is fed to a reactor in a hydroformylation zone operating under a lower carbon monoxide partial pressure.

11. The process of claim 1, further comprising a step of feeding each separate aldehyde product stream into a single N and I isomer separation zone, and recovering a high N:I ratio stream comprising aldehyde having an N:I ratio greater than N:I ratio R_{A} and a low N:I ratio stream comprising aldehyde having an N:I ratio less than N:I ratio R_{A}.

12. A process for preparing an alkylalcohol, the process comprising producing high and low N:I ratio streams using the process of claim 11, then performing an aldol condensation reaction on one of the high or low N:I ratio streams, followed by dehydration and hydrogenation steps to provide the alkylalcohol.

13. The process of claim 1 wherein olefin is propylene, and the aldehyde is butyraldehyde.

14. A process for preparing an alcohol, the process comprising the steps of producing N and I aldehydes at an N:I ratio R_{A} using the process of claim 1, then hydrogenating at least some of the aldehyde to provide the alcohol.

15. The process of claim 14, wherein the alcohol is butanol.
